(19) United States
(12) Patent Application Publication
(10) Pub. No.: US 2024/0082172 A1
(43) Pub. Date: Mar. 14, 2024

(54) METHOD FOR DEPOT CREATION DURING TRANSDERMAL DRUG DELIVERY

(71) Applicant: Corium, Inc., Menlo Park, CA (US)

(72) Inventors: Amit K. JAIN, Milpitas, CA (US); Eun Soo LEE, Redwood City, CA (US); Parminder SINGH, Union City, CA (US)

(21) Appl. No.: 18/341,541

(22) Filed: Jun. 26, 2023

Related U.S. Application Data
(63) Continuation of application No. 16/219,859, filed on Dec. 13, 2018, now abandoned.
(60) Provisional application No. 62/598,256, filed on Dec. 13, 2017.

(51) Int. Cl. A61K 9/70; A61K 31/40; A61K 31/445; A61K 47/12; A61K 47/14; A61K 47/30

(52) U.S. Cl. CPC A61K 9/7084; A61K 9/7053; A61K 9/7061; A61K 31/40; A61K 31/445; A61K 47/12; A61K 47/14; A61K 47/30

(57) ABSTRACT

Methods, compositions, and devices for transdermally administering an active agent such as donepezil are provided. In one aspect, the method comprises contacting skin with a transdermal device designed to create a depot of the active agent in the subject, removing the transdermal device and continuing to administer the active agent for a period after the device is removed.

### CROSS-REFERENCE TO RELATED APPLICATIONS

This application is a Continuation of Non-Provisional application Ser. No. 16/219,859, filed Dec. 13, 2018, which claims the benefit of U.S. Provisional Application No. 62/598,256, filed Dec. 13, 2017, incorporated by reference herein.

### TECHNICAL FIELD

The subject matter described herein relates to compositions, devices and methods for the transdermal administration of a therapeutic agent by a combined approach of transdermal delivery and depot delivery.

### BACKGROUND

Donepezil is an acetylcholinesterase inhibitor with the chemical structure 2,3-Dihydro-5,6-dimethoxy-2-[[1-(phenylmethyl)-4-piperidinyl]methyl]-1H-inden-1-one:

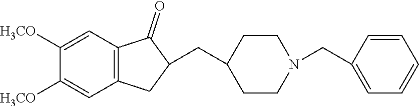 [SMILES: COc1cc2c(cc1OC)C(=O)C(CC1CCN(Cc3ccccc3)CC1)C2]

Donepezil has a molecular weight of 379.5 and is lipophilic (Log P value 3.08-4311). U.S. Pat. No. 4,895,841 to Eisai Co., Ltd. describes cyclic amine compounds including donepezil for use in treating dementia including Alzheimer senile dementia, Huntington's chorea, Pick's disease, and ataxia. An oral table of donepezil hydrochloride (Aricept®) is approved in the U.S. for use in treating Alzheimer's dementia. Oral donepezil may be associated with adverse cholinergic effects related to the gastrointestinal system including nausea, vomiting and diarrhea as well as sleep disturbance. Further, oral administration of donepezil is associated with frequent plasma fluctuations. Oral donepezil is rapidly absorbed and peak plasma levels (Cₘₐₓ) are reached in about 3 hours (Tiseo et al., Br J Clin Pharmacol, 1998, 46(Suppl 1):13-18). Due to the nature of cognitive disorders, oral medications may be subject to problems with patient compliance especially for formulations that need to be taken throughout the day.

Delivery of medications by transdermal, injection, or rectal (suppositories) routes to patients suffering from cognitive disorders has been investigated. U.S. Pat. No. 7,858,114 describes a percutaneous absorption preparation of donepezil for use as a plaster for long term delivery of an anti-dementia drug. U.S. Patent No. 2014/0370076 describes a transdermal drug delivery system comprising donepezil or a salt thereof that uses an acrylate-rubber hybrid adhesive that is prepared by a process without n-hexane. Other transdermal delivery systems proposed use an overlay or other rate-limiting membrane to control delivery of the drug from the transdermal device, see e.g. U.S. Published Application No. 2010/0178307 which describes the use of a first and second overlay. Despite these teachings, there are no donepezil transdermal patches or devices available in the United States.

Delivery of anti-dementia drugs over a long period of time (e.g. several days or more) is difficult. Transdermal delivery of basic drugs including donepezil can be especially difficult due to poor skin permeability. Further, some active agents have poor or low solubility in the adhesives and/or other components used in typical transdermal formulations. Further, there is a need for stable, long term administration of anti-dementia agents (e.g. 1-10 days or more) that provides a stable and effective release of the agent over the administration period and has suitable adhesion for the long term administration.

Depot injection formulations may provide slow release and gradual absorption. Published PCT No. WO 2013/078608 describes an intramuscular formulation comprising a pamoate salt of donepezil.

There exists a need for transdermal compositions, devices and methods that provide consistent and extended delivery of donepezil.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

Methods and compositions to effect transdermal delivery of an active agent, such as donepezil, are provided.

In a first aspect, a composition useful for a transdermal delivery system is provided. In one embodiment, the composition comprises an adhesive matrix comprising at least about 5-50 wt % donepezil; at least about 5-80 wt % of an adhesive polymer, and at least about 5-20 wt % of a solubility enhancer.

In embodiments, the solubility enhancer is selected from a dibasic ester, an ester of a dicarboxylic acid, and a dialkylsulfoxide. In other embodiments, the solubility enhancer is selected from dimethyl succinate and diethyl succinate.

In one embodiment, the adhesive matrix further comprises at least about 1-20 wt % of an permeation enhancer comprising a carboxyl group. In embodiments, the permeation enhancer is selected from a fatty acid, an α-hydroxy acid, a β-hydroxy acid, and a keto carboxylic acid.

In one embodiment, the fatty acid is a saturated fatty acid or an unsaturated fatty acid. In other embodiments, the fatty acid is a saturated fatty acid with between 16-20 carbon atoms, or an unsaturated fatty acid with between 16-20 carbon atoms. In other embodiments, the fatty acid is selected from oleic acid (C18), linoleic acid (C18) and linolenic acid (C18).

In other embodiments, the permeation enhancer is a keto acid (a compound with a carboxylic acid and a ketone group). In an embodiment, the permeation enhancer is levulinic acid.

In embodiments, the adhesive polymer is selected from an acrylic polymer, an acrylate polymer, an acrylic ester polymer, polymers comprising a pyrrolidone group, polyisobutylenes, polybutenes, polymers comprising an acetate group, derivatives and co-polymers thereof. In some embodiments, the adhesive polymer is selected from a cross-linked polyvinylpyrrolidone, a soluble polyvinylpyrrolidone, and co-polymers thereof. In some embodiments, the adhesive polymer is a mixture of polyisobutylene and polybutene polymers where the mixture is present in the composition at about 35-80 wt %.

In embodiments, the adhesive matrix further comprises at least about 1-10 wt % of a matrix modifier. In some embodiments, the matrix modifier is selected from the group consisting of fumed silica, colloidal silicon dioxide, a cellulose derivative, polyacrylamide, polyacrylic acid, a polyacrylic acid salt, kaolin, bentonite and combinations thereof.

In embodiments, the adhesive matrix further comprises a second permeation enhancer. In some embodiments, the second permeation enhancer is selected from methyl laurate, propylene glycol monolaurate, glycerol monolaurate, glycerol monooleate, lauryl lactate, laurydone, and myristyl lactate.

In another aspect, a composition for preparing a patch for transdermal delivery of donepezil is provided. In embodiments, the composition comprises an adhesive matrix comprising at least about 5-50 wt % donepezil; at least about 5-80 wt % of an adhesive polymer, and at least about 5-20 wt % of dimethyl succinate.

In some embodiments, the composition comprises an adhesive matrix comprising about 5-50 wt % donepezil; about 2-6 wt % lauryl lactate or lauryl pyrrolidone carboxylate; about 5-30 wt % dimethyl succinate; about 0-15% dimethyl sulfoxide; about 1-10 wt % levulinic acid or lactic acid; about 3-20 wt % fumed silica and/or cross-linked polyvinyl pyrrolidone; and about 35 to 80 wt % of an adhesive comprising a mixture of polyisobutylene and polybutene or a hydrogenated polybutene.

In a further aspect, a transdermal delivery device is provided. In embodiments, the transdermal delivery device comprises a backing layer, an adhesive layer comprising at least about 5-50 wt % donepezil, at least about 5-80 wt % of least one adhesive polymer, and at least about 5-20 wt % of a solubility enhancer, and a release liner. In embodiments, the transdermal delivery patch provides donepezil in a depot form.

In embodiments, the solubility enhancer is selected from a dibasic ester, an ester of a dicarboxylic acid, and a dialkylsulfoxide. In some embodiments, the ester of a dicarboxylic acid is selected from dimethyl succinate and diethyl succinate. In some embodiments, the solubility enhancer is dimethyl succinate.

In embodiments, the adhesive layer further comprises at least about 1-20 wt % of a permeation enhancer. In some embodiments, the permeation enhancer is selected from a fatty acid, an α-hydroxy acid, a β-hydroxy acid, and a keto carboxylic acid. In some embodiments, the fatty acid is selected from an unsaturated fatty acid and a saturated fatty acid.

In embodiments, the release liner is a silicone coated material. In some embodiments, the release liner is a silicone coated PET, fluorocarbon, or fluorocarbon coated PET.

In embodiments, the adhesive layer further comprises a second permeation enhancer. In some embodiments, the second permeation enhancer is selected from methyl laurate, propylene glycol monolaurate, glycerol monolaurate, glycerol monooleate, lauryl lactate, laurydone, myristyl lactate and lauryl pyrrolidone carboxylate.

In embodiments, the adhesive layer further comprises a matrix modifier. In some embodiments, the matrix modifier is selected from the group consisting of fumed silica, colloidal silicon dioxide, cross-linked polyvinylpyrrolidone, soluble polyvinylpyrrolidone, a cellulose derivative, polyacrylamide, polyacrylic acid, a polyacrylic acid salt, kaolin, bentonite and combinations thereof. In embodiments, the adhesive polymer comprises at least one of a polyacrylate, a polymethacrylate derivative, a mixture of a polyisobutylene and a polybutene polymer, and copolymers thereof.

In yet another aspect, a method for extended and continuous release of donepezil is provided. The donepezil can be in base form or in salt form. The method comprises applying to a first skin site on a subject an adhesive matrix comprising between about 5-50 wt % donepezil and between about 5-40 wt % of an ester of a dicarboxylic acid; allowing the adhesive matrix to remain on the skin for about 24 hours to create a first donepezil depot in the subject; removing the adhesive matrix from the skin; and continuing to administer donepezil from the donepezil depot after the adhesive matrix is removed.

In one embodiment, the method further comprises contacting skin of the subject with a second transdermal device comprising an adhesive matrix comprised of between about 5-50 wt % donepezil and between about 5-40 wt % of dimethyl succinate.

In one embodiment, contacting skin with a second transdermal device comprises contacting at the first skin site. In other embodiments, contacting skin with a second transdermal device comprises contacting at a second skin site that is different from the first skin site.

In an embodiment, contacting at a second skin site creates a second donepezil depot.

In yet another embodiment, the second donepezil depot is created before exhaustion of the first donepezil depot.

In another embodiment, the first donepezil depot is created in the skin of the subject.

In another embodiment, contacting with a second transdermal device is performed during the period of donepezil administration from the first donepezil depot.

In still another embodiment, the ester of a dicarboxylic acid is selected from dimethyl succinate and diethyl succinate.

The adhesive matrix in other embodiments further comprises between about 1-20 wt % of a permeation enhancer selected from a fatty acid, an α-hydroxy acid, a β-hydroxy acid, and a keto carboxylic acid.

In an embodiment, the permeation enhancer is a fatty acid selected from oleic acid, linoleic acid, linolenic acid, and levulinic acid.

In still another aspect, a method for administering donepezil is provided. The method comprises contacting skin of a subject with a transdermal device comprising an adhesive matrix comprised of between about 5-50 wt % donepezil base and between about 5-40 wt % of dimethyl succinate; allowing the adhesive matrix to remain on the skin for a time sufficient to create a donepezil depot in the skin; removing the transdermal device from the skin; and continuing to administer donepezil from the donepezil depot after said removing for a period of at least about 6 hours.

In one embodiment, the method further comprises contacting skin of the subject with a second transdermal device comprising an adhesive matrix comprised of between about 5-50 wt % donepezil base and between about 5-40 wt % of dimethyl succinate, where the second transdermal device is contacted at a same or a different site on the skin.

In one embodiment, the step of contacting with a second transdermal device is performed during the period of donepezil administration from the donepezil depot.

In another embodiment, the method comprises continuing to administer donepezil from the donepezil depot after removal of the transdermal device is for a period of between about 6-48 hours.

In one embodiment, continuing to administer provides a therapeutically effective amount of donepezil to the subject for at least 25%, 50%, 65% or 75% of the period.

In another embodiment, allowing the adhesive matrix to remain on the skin for a time sufficient to create a donepezil depot in the skin comprises a time of at least about 12 hours.

In still another embodiment, the time sufficient to create a donepezil depot is between about 12-48 hours.

In another aspect, a method of making a patch for the transdermal delivery of donepezil is provided. In embodiments, the method comprises preparing a donepezil solution by dissolving donepezil base in a mixture of one or more solubility enhancers and a suitable solvent; preparing an adhesive polymer solution by (i) dissolving or dispersing one or more hydrophilic polymers in the donepezil solution of (a) and/or dissolving one or more hydrophobic polymers in a suitable solvent and mixing the hydrophobic polymer solution with the donepezil solution of (a) to form a homogenous or dispersed mixture; coating the adhesive drug formulation on a silicone-coated polyethylene terephthalate film; and drying the coated film.

In embodiments, the solubility enhancer is dimethyl succinate.

In embodiments, the hydrophilic polymer is dissolved or dispersed in the solvent at a concentration of about 4 to 35% w/w. In embodiments, the hydrophobic polymer is selected from a mixture of polyisobutylene and polybutene polymers, and an acrylic ester polymer.

In embodiments, the method further comprises applying a backing layer onto the adhesive layer opposite the silicone-coated PET film layer.

In a further aspect, a method of improving cognition is provided. In embodiments, the method comprises applying a transdermal patch to the skin of a patient in need thereof, where the patch comprises an adhesive matrix comprises (a) at least about 5-50 wt % donepezil; (b) at least about 5-80 wt % of an adhesive polymer, and (c) at least about 5-20 wt % of dimethyl succinate; wherein the patch delivers donepezil to the patient as a depot. In embodiments, the depot delivers donepezil for a period of at least six hours after the patch is removed.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIGS. 1A-1B are illustrations of transdermal patch configurations in some embodiments.

FIG. 2 is a graph of the average skin flux for a donepezil transdermal delivery device μg/cm²·hr in vitro as a function of time, in hours.

### DETAILED DESCRIPTION

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

The present compositions, devices, and methods are not limited to the specific polymers, excipients, cross-linking agents, additives, manufacturing processes, or adhesive products described herein. It will be understood that the particular terminology used herein is for the purpose of describing particular embodiments and is not intended to be limiting.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The use of terms of order or importance, including “first” and “second”, is to distinguish and identify individual elements and does not denote or imply a particular order or importance unless clearly indicated by context.

An “adhesive matrix” as described herein includes matrices made in one piece, for example, matrices made via solvent casting or extrusion as well as matrices formed in two or more portions that are then pressed or joined together.

The term “skin” as used herein refers to skin or mucosal tissue, including the interior surface of body cavities that have a mucosal lining. The term “skin” should be interpreted as including “mucosal tissue” and vice versa.

“Donepezil” as used herein refers to 2,3-dihydro-5,6-dimethoxy-2-[[1-(phenylmethyl)-4-piperidinyl]methyl]-1H-inden-1-one.

The term “therapeutically effective amount” as used herein refers to the amount of an active agent that is nontoxic but sufficient to provide the desired therapeutic effect. The amount that is “effective” will vary from subject to subject, depending on the age and general condition of the individual, the particular active agent or agents, and the like as known to those skilled in the art.

The terms “transdermal” or “transdermal delivery” as used herein refer to administration of an active agent to a body surface of an individual so that the agent passes through the body surface, e.g., skin, and into the individual's blood stream. The term “transdermal” is intended to include transmucosal administration, i.e., administration of a drug to the mucosal (e.g., sublingual, buccal, vaginal, rectal) surface of an individual so that the agent passes through the mucosal tissue and into the individual's blood stream.

### II. Compositions and Transdermal Delivery Devices

The compositions and devices described herein are designed for transdermal administration of an active agent such as donepezil. The compositions may be used in devices, patches or systems suitable for transdermal delivery of the agent. The active agent is discussed with reference to donepezil below. However, it will be appreciated that the discussion is applicable to other suitable active agents.

One impediment to transdermal delivery is that many active agents such as donepezil have low solubility in the adhesive matrix. In embodiments, the present compositions are suitable for active agents having low solubility, that is, a solubility of less than about 3 wt % in a rubber or silicone adhesive. In some embodiments, the active agent has a solubility of least about 10 wt % in the composition. The composition comprises an adhesive or adhesive matrix that comprises the active agent, a lipophilic penetration enhancer, and a solubilizing agent to increase solubility of the therapeutic agent in the adhesive matrix and/or prevent precipitation of the therapeutic agent in the adhesive matrix. In preferred embodiments, the active agent is donepezil in free base form. In some embodiments, the donepezil free base is in non-crystalline form. In some embodiments, the composition comprises an adhesive matrix comprising the active agent, one or more adhesive polymers, a dibasic ester, and an acid. In some embodiments, the adhesive matrix comprises about 5-50 wt % of the active agent. In some embodiments, the adhesive matrix comprises about 5-40 wt %, 5-30 wt %, 5-25 wt %, 5-20 wt %, 5-15 wt %, 5-10 wt %, 10-50 wt %, 10-40 wt %, 10-30 wt %, 10-25 wt %, 10-20 wt %, 10-15 wt %, 20-50 wt %, 20-40 wt %, 20-30 wt %, 20-25 wt %, 30-50 wt %, 30-40 wt %, or 40-50 wt % of the active agent.

The adhesive matrix comprises one or more polymers. In embodiments, one or more of the polymers is an adhesive polymer. In embodiments, the adhesive matrix comprises at least about 25-80 wt % of polymers relative to the weight of the adhesive matrix (inclusive of sub-ranges). In embodiments, the matrix comprises at least about 35-80%, 30-75%, at least about 40-75%, at least about 50-75%, at least about 60-75%, at least about 25-70%, at least about 30-70%, at least about 40-70%, at least about 50-70%, at least about 60-70%, at least about 25-60%, at least about 30-60%, at least about 40-60%, at least about 50-60%, at least about 25-50%, at least about 30-50%, at least about 40-50%, at least about 25-40%, at least about 30-40%, or at least about 25-30% of the polymers (all percentages in wt %). It will be appreciated that the adhesive matrix may include one or more or at least one polymers. In embodiments, the adhesive matrix comprises at least about 5-75% of an individual polymer relative to the total weight of the polymers in the matrix. In embodiments, the adhesive matrix comprises at least about 5-10%, 5-15%, 5-20%, 5-25%, 5-30%, 5-40%, 5-50%, 5-60%, 5-70%, 5-75%, 10-15%, 10-20%, 10-20%, 10-25%, 10-30%, 10-40%, 10-50%, 10-60%, 10-70%, 10-75%, 15-20%, 15-25%, 15-30%, 15-40%, 15-50%, 15-60%, 15-70%, 15-75%, 20-25%, 20-30%, 20-40%, 20-50%, 20-60%, 20-70%, 20-75%, 25-30%, 25-40%, 25-50%, 25-60%, 25-70%, 25-75%, 30-40%, 30-50%, 30-60%, 30-70%, 30-75%, 40-50%, 40-60%, 40-70%, 40-75%, 50-60%, 50-70%, 50-75%, 60-70%, 60-75%, or 70-75% of an individual polymer.

In one embodiment, the matrix is comprised of one or more pressure sensitive adhesive polymers. In embodiments, the adhesive polymer is an acrylic polymer. In some embodiments, the adhesive polymer is an acrylic pressure sensitive adhesive polymer. In embodiments, the acrylic polymer is a polyacrylate adhesive polymer. An acrylic pressure sensitive adhesive polymer is a polyacrylate that is a polymer of copolymer of a monomer or monomers selected from acrylic acid esters and methacrylic acid esters. Other monomers, such as acrylic acid and vinyl acetate, may be present. In embodiments, the acrylic polymer is based on acrylic esters such as 2-ethylhexyl acrylate (2-EHA) and ethyl acrylate. In some embodiments, the polyacrylate polymer is a polymer or a copolymer of a monomer or monomers selected from acrylic acid and vinyl acetate. In embodiments, the acrylic polymer adhesive has pendent carboxyl (—COOH) or hydroxyl (—OH) functional groups. In embodiments, the acrylic polymer adhesive comprises at least one of polyacrylate, polymethacrylate, derivatives thereof, and co-polymers thereof. In embodiments, the acrylic adhesive is comprised of an acrylate copolymer comprising acrylic ester monomers or vinyl acetate monomers. Exemplary acrylate copolymers are sold under the trade-name DURO-TAK and include, but are not limited to, DURO-TAK 387-2516, 387-2051, and 387-2074.

In some embodiments, the matrix is comprised of one or more polymers comprising a pyrrolidone group, polyisobutylenes, polybutenes, mixtures and co-polymers thereof. In embodiments, the adhesive matrix comprises a blend or mixture of polyisobutylene and polybutene polymers. Polyisobutylene is a vinyl polymer comprised of the isobutylene monomer. Polybutene is a viscous, non-drying, liquid polymer, prepared by copolymerization of 1- and 2-butene with a small quantity of isobutylene. In some embodiments, the polybutene in one embodiment has a molecular weight of between about 750-6000 Daltons, preferably between about 900-4000 Daltons, and preferably between about 900-3000 Daltons. In some embodiments the mixture comprises polybutene in the polyisobutylene blend at about 40 weight percent. More generally, the polybutene is present in the polyisobutylene blend in an amount between 20-50 weight percent, or between 25-45 weight percent. In some embodiments, the adhesive matrix does not include an acrylate-rubber adhesive.

In embodiments, the copolymer is selected from a polyvinylpyrrolidone/vinyl acetate copolymer, an acrylic acid/vinyl acetate copolymer, and a vinyl acetate/ethylene acetate copolymer. In one embodiment, the copolymer is a vinyl acetate/N-vinylpyrrolidone copolymer such as the copolymer sold as Plasdone™ S630 (Ashland). In another embodiment, the polyvinylpyrrolidone-vinyl acetate copolymer is a linear random copolymer of n-vinyl-2-pyrrolidone and vinyl acetate. In one embodiment, the copolymer is a 60:40 copolymer of n-vinyl-2-pyrrolidone and vinyl acetate. In exemplary embodiments, the acrylic polymer is a pressure sensitive adhesive such as the polymers and copolymers sold as DURO-TAK™. In specific, but not limiting embodiments, the matrix comprises at least one acrylate co-polymer selected from DURO-TAK™ nos. 387-2516, 387-2051 and 387-2074. In embodiments, the adhesive matrix comprises polyvinylpyrrolidone (PVP). PVP is a water-soluble polymer comprised of the N-vinylpyrrolidone monomer.

In some embodiments, the adhesive matrix comprises hydrophilic and hydrophobic polymers. One skilled in the art may determine suitable hydrophilic and/or hydrophobic polymers by means known in the art. By way of example, a polyvinylpyrrolidone-vinyl acetate copolymer is a hydrophilic polymer and each of polyisobutylene, polyisobutylene and polybutene mixture, and an acrylic acid/vinyl acetate copolymer are hydrophobic. In embodiments, the polymers may include a cross-linker.

In embodiments, the adhesive matrix comprises a lipophilic permeation enhancer. In some embodiments, the permeation enhancer is an acid. In embodiments, the permeation enhancer is selected from fatty acids, fatty alcohol esters, α-hydroxy acids, β-propionic acids, dibasic esters, and esters of dicarboxylic acids. It will be appreciated that the adhesive matrix may include a permeation enhancer. In some embodiments, the adhesive matrix comprises at least two permeation enhancers.

In some embodiments, the fatty acid is a C₅-C₂₀ fatty acid. In some embodiments, the fatty acid is a C₅-C₈ or a C₈-C₂₀ fatty acid. The fatty acid may be a saturated or an unsaturated fatty acid. In some embodiments, the fatty acid is selected from one or more of valeric acid, caprylic acid, capric acid, lauric acid, myristic acid, palmitic acid, stearic acid, oleic acid, linoleic acid, linolenic acid, levulinic acid, and arachidic acid.

In some embodiments, the fatty alcohol ester has the chemical formula CH₃(CH₂)ₘCOOR′ or CH₃(CH₂)ₘ—OCOCHR₁R₂ where m is an integer between 8 and 14. R′ is a lower C₁-C₃ alkyl residue that is unsubstituted or substituted with 1, 2 or 3 hydroxyl groups, and R₁ and R₂ are individually hydrogen, hydroxyl, or a lower C₁-C₂ alkyl. In some embodiments, one of R₁ and R₂ is hydrogen and the other is hydroxyl. In other embodiments, one of R₁ and R₂ is hydroxyl and the other is a lower C₁-C₂ alkyl.

In some embodiments, the α-hydroxy acid is selected from lactic acid and glycolic acid. It will be appreciated that the fatty acid, α-hydroxy acid, and/or β-propionic acids may be any suitable acid that is in liquid form at room temperature and is safe for transdermal administration as known in the art.

The lipophilic penetration enhancer is included in the adhesive matrix in an amount to provide sufficient transport of at least the active agent across the skin. The amount of lipophilic penetration enhancer, as well as additional penetration or permeation enhancers, may be determined based on the permeability of the active agent. In embodiments, the lipophilic penetration enhancer is included in an amount about between about 1-15% relative to the weight of the adhesive matrix (inclusive of sub-ranges). In embodiments, the lipophilic penetration enhancer is included in an amount of between about 1-5%, 1-10%, 2-5%, 2-10%, 2-15%, 3-5%, 3-10%, 3-15%, 4-5%, 4-10%, 4-15%, 5-10%, 5-15%, 10-15%, etc. relative to the weight of the adhesive matrix (inclusive of sub-ranges).

The solubilizing agent or solubility enhancing agent may be used to increase solubility of the active agent in the adhesive matrix and/or to prevent precipitation of the agent in the adhesive matrix. In embodiments, the solubilizing agent or solubility enhancing agent is a dibasic ester. In some embodiments, the dibasic ester is a dicarboxylic acid. In some embodiments, the dibasic ester is an ester of succinic acid. In some embodiments, the ester of succinic acid is one of dimethyl succinate or diethyl succinate. In embodiments, the solubilizing agent or solubility enhancing agent is present in the adhesive matrix in an amount (by weight) between about 5-50%, about 5-40%, 5-30%, 5-25%, 5-20%, 5-15%, 5-9.5%, 6-16 wt %, 6-12 wt %, 6-9.5 wt %, 7-30%, 7-25%, 7-20%, 7-15%, 7-12%, 7-9.5 wt % 8-30%, 8-25%, 8-20%, 8-15%, 8-12%, 9-30%, 9-25%, 9-20%, 9-15%, 9- 12%, 10-50%, 10-40%, 10-30%, 10-25%, 10-20%, 10-15%, 15-50%, 15-40%, 15-30%, 15-25%, 15-20%, 20-50%, 20-40%, 20-30%, 20-25%, 25-50%, 25-40%, 25-30%, 30-50%, 30-40%, or 40-50%. In one embodiment, the solubility enhancing agent is present in the adhesive matrix in an amount of less than 10 wt %. In other embodiments, the solubility enhancing agent is present in the adhesive matrix in an amount greater than 1 wt % and less than 10 wt %. In some embodiments, the amount is between 1-9.8 wt %, 1-9.5 wt %, 1.5-9.8 wt %, 1.5-9.5 wt %, 2-9.8 wt %, 2-9.5 wt %, 2.5-9.8 wt %, 2.5-9.5 wt %, 3-9.8 wt %, 3-9.5 wt %, 3.5-9.8 wt %, 3.5-9.5 wt %, 4-9.8 wt %, 4.5-9.5 wt %, 5-9.8 wt %, 5-9.5 wt %, 5.5-9.8 wt %, 5.5-9.5 wt %, 6-9.8 wt %, 6-9.5 wt %, 7-9.8 wt %, or 7-9.5 wt %.

In some embodiments, the composition includes one or more additives or excipients including, but not limited to, additional penetration or permeation enhancers and/or matrix modifiers.

In some embodiments, a penetration or permeation enhancer (in addition to the lipophilic penetration or permeation enhancer described above) is included in the adhesive matrix. In embodiments, the additional penetration or permeation enhancer is a pyrrolidone compound comprising a 5-membered lactam. In some embodiments, the pyrrolidone compound is selected from one or more of laurydone, a lauric acid ester of pyrrolidone, and pyrrolidone derivatives having a fatty acid chain of at least 5-8 carbons or a fatty acid chain that is longer than 8 carbons.

The penetration or permeation enhancer may be chosen from a wide range of such compounds known in the art. In some embodiments, permeation enhancers for use in the adhesive matrix include, but are not limited to, methyl laurate, propylene glycol monolaurate, glycerol monolaurate, glycerol monooleate, lauryl lactate, myristyl lactate, and dodecyl acetate. Additional permeation enhancers are described in U.S. Pat. No. 8,874,879, which is incorporated herein by reference. It will be appreciated that the compositions herein may include one or more or at least one permeation enhancer. In embodiments, the penetrating or permeating enhancer is included in an amount between about 1-10%, about 2-5%, about 2-10% relative to the weight of the adhesive matrix (inclusive of sub-ranges).

The permeation enhancers may be used to adjust the rate of delivery and/or dosage administered. The dosage may be affected by the rate of permeation from the patch into the blood stream and/or the size (e.g. cm²) of the transdermal device.

The adhesive matrix may further include one or more matrix modifiers. Without wishing to be bound by theory, it is believed that the matrix modifier facilitates homogenization of the adhesive matrix. Sorption of hydrophilic moieties is a possible mechanism for this process. Thus, known matrix modifiers which are to some degree water-sorbent may be used. For example, possible matrix modifiers include colloidal silicone dioxide, fumed silica, cross-linked polyvinylpyrrolidone (PVP), soluble PVP, cellulose derivatives (e.g. hydroxypropyl cellulose (HPC), hydroxyethylcellulose (HEC)), polyacrylamide, polyacrylic acid, a polyacrylic acid salt, or a clay such as kaolin or bentonite. An exemplary commercial fumed silica product is CAB-O-SIL® (Cabot Corporation, Boston. Mass.). The hydrophilic mixtures described in U.S. Published Patent Application No. 2003/0170308 may also be employed, for example mixtures of PVP and PEG or of PVP, PEG, and a water-swellable polymer such as Eudragit® L100-55. In embodiments, the matrix modifier is individually included in an amount between about 1-25%, about 2-25%, about 5-25%, about 5-7%, about 7-20%, or about 7-25% relative to the weight of the adhesive matrix (inclusive of sub-ranges). In some embodiments, the matrix modifier does not include ethylcellulose.

The composition may also include other conventional additives such as adhesive agents, antioxidants, crosslinking or curing agents, pH regulators, pigments, dyes, refractive particles, conductive species, antimicrobial agents, opacifiers, gelling agents, viscosity modifiers or thickening agents, stabilizing agents, and the like as known in the art. In those embodiments wherein adhesion needs to be reduced or eliminated, conventional detackifying agents may also be used. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. These additives, and amounts thereof, are selected in such a way that they do not significantly interfere with the desired chemical and physical properties of the adhesive and/or active agent.

The compositions may also contain irritation-mitigating additives to minimize or eliminate the possibility of skin irritation and/or skin damage resulting from the drug, the enhancer, or other components of the composition. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. Corticosteriods are also known in art as irritation-mitigating additives.

Methods for preparing or manufacturing the adhesive matrix are also provided. With reference to Example 1, in embodiments, the methods comprise (i) solubilizing one or more polymers in a suitable solvent or solvents, (ii) mixing (i) with at least one solubility enhancer and at least one lipophilic permeation enhancer, (iii) dissolving at least about 5% w/w of an active agent such as donepezil free base to (ii), and forming an adhesive matrix from the adhesive solution that comprises at least about 5-50 wt % of at least one adhesive polymer, at least about 5-50 wt %, 5-40 wt %, 5-35 wt %, or 10-30% wt % of a solubility enhancer, and between about 1-20 wt %, 1-15 wt %, 1-10 wt %, 2-15 wt %, 2-10 wt %, 3-15 wt % or 4-10 wt % of a lipophilic penetration enhancer.

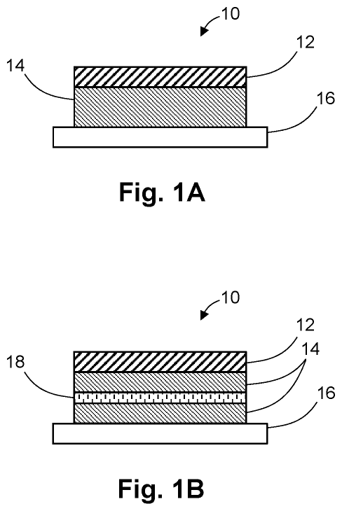

The adhesive matrix may be used as an adhesive layer in a transdermal device, apparatus, or patch. Some exemplary devices are shown in FIGS. 1A-1B. As seen in FIG. 1A, a transdermal device 10 includes a backing layer 12, an adhesive matrix layer 14 and an optional release liner 16. FIG. 1B is a schematic of another exemplary device comprising two adhesive matrix layers separated by a tie layer or a rate controlling membrane layer 18. In this embodiment, one or both of the adhesive matrix layers 14 may contain drug, and in one embodiment, the adhesive matrix layer adjacent the backing layer is a drug-containing adhesive matrix and the adhesive matrix layer adjacent the release layer is manufactured from a composition that is drug free.

In some embodiments, the backing layer provides a structural element for holding or supporting the adhesive layer. The backing layer may be formed of any suitable material as known in the art. In some embodiments, the backing layer is occlusive. In some embodiments, the backing is preferably impermeable or substantially impermeable to moisture. In one exemplary embodiment, the barrier layer has an MVTR (moisture vapor transmission rate) of less than about 50 g/m²-day. In some embodiments, the backing layer is preferably inert and/or does not absorb components of the adhesive layer, including the active agent. In some embodiments, the backing layer preferably prevents release of components of the adhesive layer through the backing layer. The backing layer may be flexible or nonflexible. The backing layer is preferably at least partially flexible such that the backing layer is able to conform at least partially to the shape of the skin where the patch is applied. In some embodiments, the backing layer is flexible such that the backing layer conforms to the shape of the skin where the patch is applied. In some embodiments, the backing layer is sufficiently flexible to maintain contact at the application site with movement, e.g. skin movement. Typically, the material used for the backing layer should permit the device to follow the contours of the skin or other application site and be worn comfortably on areas of skin such as at joints or other points of flexure, that are normally subjected to mechanical strain with little or no likelihood of the device disengaging from the skin due to differences in the flexibility or resiliency of the skin and the device.

In some embodiments, the backing layer is formed of one or more of a film, non-woven fabric, woven fabric, laminate, and combinations thereof. In some embodiments, the film is a polymer film comprised of one or more polymers. Suitable polymers are known in the art and include elastomers, polyesters, polyethylene, polypropylene, polyurethanes and polyether amides. In some embodiments, the backing layer is formed of one or more of polyethylene terephthalate, various nylons, polypropylene, metalized polyester films, polyvinylidene chloride, and aluminum foil. In some embodiments, the backing layer is a fabric formed of one or more of polyesters such as polyethylene terephthalate, polyurethane, polyvinyl acetate, polyvinylidene chloride and polyethylene. In one particular, but non-limiting embodiment, the backing layer is formed of a polyester film laminate. One particular polyester film laminate is the polyethylene and polyester laminate such as the laminate sold under the name Scotchpak™ #9723.

The device includes at least one adhesive layer adjacent the backing layer. In embodiments, the adhesive layer is an adhesive matrix comprising the active agent as described above. The adhesive layer adheres to the backing layer and/or skin at the administration site. The adhesive layer matrix also serves to release the active agent to the skin.

In embodiments, the device includes a release liner at least partially in contact at least with the adhesive layer to protect the adhesive layer prior to application. The release liner is typically a disposable layer that is removed prior to application of the device to the treatment site. In some embodiments, the release liner preferably does not absorb components of the adhesive layer, including the active agent. In some embodiments, the release liner preferably impermeable to components of the adhesive layer (including the active agent) and prevents release of components of the adhesive layer through the release liner. In some embodiments, the release liner is formed of one or more of a film, non-woven fabric, woven fabric, laminate, and combinations thereof. In some embodiments, the release liner is a silicone-coated polymer film or paper. In some non-limiting embodiments, the release liner is a silicone-coated polyethylene terephthalate (PET) film, a fluorocarbon film, or a fluorocarbon coated PET film.

In some embodiments, the device further includes a fabric or tie layer 18 within the adhesive matrix. The tie layer may be formed of any suitable material including, but not limited to, polyesters, vinyl acetate polymers and copolymers, polyethylenes, and combinations thereof. In one embodiment, the tie layer is a nonwoven layer of polyester fibers such as the film sold under the name Reemay® (Kavon Filter Products Co.). In embodiments, the tie layer does not affect the rate of release of the active agent from the adhesive layers.

The thickness and/or size of the device and/or adhesive matrix may be determined by one skilled in the art based at least on considerations of wearability and/or required dose. It will be appreciated that the administration site for the device will affect the wearability considerations due to the available size of the administration site and the use of the administration site (e.g. need for flexibility to support movement). In some embodiments, the device and/or adhesive matrix has a thickness of between about 25-500 μm. In some embodiments, the device and/or adhesive matrix has a thickness of between about 50-500 μm. In some embodiments, the patch has a size in the range of about 16 cm²-225 cm². It will be appreciated that the thickness and size provided here are merely exemplary and the actual thickness and or size may be thinner/smaller or thicker/larger as needed for a specific formulation.

An adhesive formulation is cast or otherwise applied to a suitable film such as a release liner and dried to remove all solvents and/or volatile compounds. In some embodiments, the formulation is dried at a temperature between about 5-100° C. The adhesive matrix is then laminated to a suitable film such as a backing layer or film.

Adhesive formulations and adhesive matrices were prepared to illustrate the embodiments described herein. Examples 1 and 2-7 set forth exemplary formulations and the resulting adhesive matrices using donepezil in free base form.

In Example 1, an adhesive formulation comprising the active agent at 10.0 wt %, an adhesive polymer at about 40 wt %, a solubility enhancer at between about 10-30 wt %; and an acid lipophilic permeation enhancer at about 3 wt %. The adhesive polymer was an acrylic acid/vinyl acetate copolymer. The adhesive matrix further included matrix modifiers and further skin penetration enhancers. The ability to use an active agent such as donepezil in its free base form allows for a lower drug load in the adhesive matrix with similar delivery as the salt forms of the drug. The formulation of Example 1 comprises an ester of a dicarboxylic acid (dimethyl succinate) which is effective to prevent the active agent from precipitating in the polymers. The solubility enhancer also provides a matrix for the drug to dissolve and remain in solution. The increased solubility of the active agent in the adhesive matrix provides greater stability and a resulting increase in shelf life. The formulation of Example 1 also includes an acid permeation enhancer (levulinic acid). The permeation enhancer forms a complex with the active agent thereby increasing the solubility of the active agent in the skin resulting in enhanced permeation of the active agent through the skin.

In Example 3, an adhesive formulation comprising the active agent at 10 wt %, adhesive polymers at about 45-55 wt %, a solubility enhancer at about 10-30 wt %; and an acid lipophilic permeation enhancer at about 3 wt %. The adhesive polymers were comprised of a homogeneous blend of an acrylic acid/vinyl acetate copolymer and a polyvinylpyrrolidone/vinyl acetate copolymer. The adhesive matrix further included matrix modifiers and further skin penetration enhancers.

In Example 4, an adhesive formulation comprising the active agent at about 10 wt %, adhesive polymers at about 50-60 wt %, a solubility enhancer at about 10-30 wt %; and an acid lipophilic permeation enhancer at about 3 wt %. The adhesive polymers were comprised of a homogeneous blend of an acrylic acid/vinyl acetate copolymer, a polyvinylpyrrolidone/vinyl acetate copolymer, and an acrylic acid copolymer. The adhesive matrix further included matrix modifiers and further skin penetration enhancers.

In Example 5, an adhesive formulation comprising the active agent at 10.0 wt % and adhesive polymers at about 56.0 wt %. The adhesive polymers were comprised of a polyisobutylene/polybutene mixture. This adhesive formulation did not include a solubility enhancer or an acid lipophilic permeation enhancer.

In Example 6, an adhesive formulation comprising the active agent at 10.0 wt %, an adhesive polymer at about 56 wt %, a solubility enhancer at 10-30 wt %; and an acid lipophilic permeation enhancer at 3.0 wt %. The adhesive polymer was comprised of a polyisobutylene/polybutene mixture. The adhesive matrix further included a matrix modifier and further skin penetration enhancers.

In Example 7, an adhesive formulation comprising the active agent at 10.0 wt %, adhesive polymers at about 56 wt %, a solubility enhancer at 10-30 wt %; and an acid lipophilic permeation enhancer at 3.0 wt %. The adhesive polymer was comprised of a polyisobutylene/polybutene mixture. The adhesive matrix further included a matrix modifier and further skin penetration enhancers.

### III. Methods of Treatment

Based on the exemplary compositions and devices described herein, and the data showing release of donepezil from the adhesive, skin-contacting layer, a method for administering donepezil to a subject in need is provided.

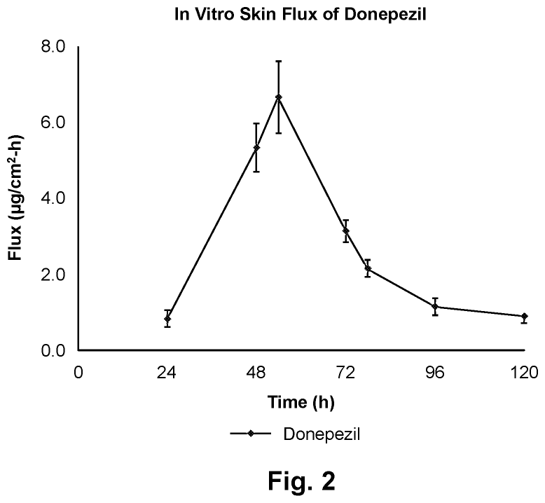

A study was performed using a transdermal delivery device comprising donepezil and a solubility enhancer in an adhesive matrix. As described in Example 8, the transdermal device comprised of an adhesive polymer, donepezil, and dimethyl succinate as a solubility enhancer was prepared. The device was tested in vitro using human cadaver skin using a standard skin permeation testing apparatus where the concentration of donepezil in the receiving fluid is measured as a function of time. The transdermal patch was applied to the cadaver skin was allowed to remain on the skin for 48 hours. The transdermal patch was then removed from the skin and the donepezil concentration was measured for another 72 hours. The data is shown in FIG. 2.

As seen in FIG. 2, the skin flux of donepezil increased during the 48 hour period while the transdermal patch was in contact with the skin, to about 6.0 μg/cm²·hr. After removal of the patch at the 48 hour time point, donepezil continued to be delivered across the skin for a period of 24-72 hours. Thus, a depot of donepezil was created during the period of patch wear, to provide a sustained and continuous delivery of donepezil for a period longer than the period the patch was in contact with the skin. In one embodiment, the sustained, continuous delivery is for at least at least about 50% of the time the patch was in skin contact. For example, if the patch is in skin contact for 48 hours, donepezil delivery continues for at least about 24 hours. In one embodiment the period of delivery from the depot is sufficient to provide a therapeutically effective amount of the drug to the subject.

In other embodiments, the sustained, continuous delivery is for at least at least about 25%, 75% or equal to the time the patch was in skin contact. In yet another embodiment, the sustained, continuous delivery is for a time that is 25%, 50% or 75% longer than the period of time the patch was in skin contact. In one embodiment, a therapeutically effective amount of the drug is provided for all or a portion of the period of delivery from the depot.

With continued reference to FIG. 2, the flux of donepezil through the skin decreased to about 2.0 μg/cm²·hr by just over 72 hours. Thus, the drug was delivered from the transdermal patch to the skin to create a depot over a first time period. In one embodiment, during the first time period, drug is administered to the subject in an amount to reach a steady state therapeutic blood concentration and to create a depot. The patch is then removed from the skin after the first time period, and drug continues to be delivered to the subject for a second time period. In one embodiment, the second time period is at least about 6 hours, and in other embodiments is between 6-48 hours, 6-24 hours, 6-18 hours, 6-12 hours, 12-48 hours, 12-24 hours or 12-18 hours. In one embodiment, the transdermal devices described herein provide a sustained, continuous and/or increasing skin flux for at least about 6 hours after the patch is removed. The depot of donepezil created in the skin provides a sustained and/or continuous delivery of donepezil after removal of the transdermal patch.

Accordingly, a method for extended and/or continuous release of donepezil or for the administration of donepezil is provided. The donepezil can be in base form or in salt form. The method comprises applying to a first skin site on a subject an adhesive matrix comprising donepezil and an ester of a dicarboxylic acid; allowing the adhesive matrix to remain on the skin for a first period. During the first period, donepezil is administered to the subject and a depot is created. The adhesive matrix is removed from the skin, and after removal donepezil continues to be administered to the subject. It will be appreciated that the adhesive matrix can be formulated to comprise the amounts of components described in the preceding sections of this document and in the working examples, and that the time periods of adhesive matrix wear and time periods of drug delivery mentioned elsewhere in this document are contemplated as part of the methods.

The method may further comprise contacting skin of the subject with a second adhesive matrix (or transdermal device or patch), the second adhesive matrix in one embodiment having the same composition of first adhesive matrix applied to the skin. The second adhesive matrix (or transdermal device or patch) can be applied to the same or a different site on the skin, to create a second depot in the same or different site as the first depot. A skilled person will appreciate that the second adhesive matrix (or transdermal device or patch) can be applied before exhaustion of the first donepezil depot, or before the first donepezil depot depletes to a level such that a therapeutically effective amount of drug is no longer provided by the depot. In one embodiment, after removal of the first or second adhesive matrix (or transdermal device or patch) donepezil is continued to be administered in an amount and/or at a rate to provide a therapeutically effective amount of donepezil to the subject for at least 25%, 50%, 65% or 75% of the period during which drug is delivered from the depot.

In another embodiment, allowing the adhesive matrix to remain on the skin for a time sufficient to create a donepezil depot in the skin comprises a time of at least about 12 hours. In still another embodiment, the time sufficient to create a donepezil depot is between about 12-48 hours.

The methods for administering donepezil are useful for treating, delaying progression, delaying onset, slowing progression, preventing, providing remission, and improvement in symptoms of cognitive disorders or disease are provided herein. In embodiments, compositions and devices comprising donepezil are provided for maintaining mental function including, but not limited to a least one of maintaining thinking, memory, speaking skills as well as managing or moderating one or more behavioral symptoms of a cognitive disorder or disease. In embodiments, the cognitive disorder is Alzheimer's disease. In particular embodiments, the cognitive disorder is Alzheimer's type dementia. In embodiments, compositions and devices comprising donepezil are provided for use in treating, etc. mild, moderate, or severe Alzheimer's disease.

Alzheimer's disease is the most common cause of senile dementia and is characterized by cognitive deficits related to degeneration of cholinergic neurons. Alzheimer's affects 6-8% of people over the age of 65 and nearly 30% of people over the age of 85 (Sozio et al., Neurophsychiatric Disease and Treatment, 2012, 8:361-368), involving the loss of cognitive functioning and behavioral abilities. The causes of Alzheimer's disease are not yet fully understood. As Alzheimer's disease is associated with reduced levels of several cerebral neurotransmitters including acetylcholine (Ach), current treatment includes administering cholinesterase inhibitors. Cholinesterase inhibitors reduce the hydrolysis of acetylcholine in the synaptic cleft by inhibiting cholinesterase and/or butyrylcholinesterase, which increases acetylcholine levels resulting in improved neurotransmission (Sozio et al.).

The transdermal devices described herein may be designed for long term use and/or continuous administration of the active agent. The FDA has approved daily oral doses of donepezil of 5 mg, 10 mg, and 23 mg. It will be appreciated that the total dose of the active agent per transdermal device will be determined by the size of the device and the loading of the active agent within the adhesive matrix. In an embodiment, the active agent is donepezil in free base form. Lower drug loading of donepezil base may be effective as compared to the salt form (e.g. donepezil hydrochloride). The ability to include lower drug loading to achieve efficacy results in a lower profile for the device (thinner) and/or smaller size, both of which are desirable to reduce discomfort. In some embodiments, the application period for the transdermal device is between about 1-10 days, 1-7 days, 1-5 days, 1-2 days, 3-10 days, 3-7 days, 3-5 days, 5-10 days, and 5-7 days inclusive. In some embodiments, the active agent is released from the adhesive matrix as a continuous and/or sustained release over the application period.

In embodiments, the transdermal devices described herein are designed to create a depot for release for administration of the active agent. In embodiments, the transdermal devices described herein provide sustained or continuous delivery of donepezil. The depot provides continuous or sustained release of the active agent after the transdermal delivery device is removed. In some embodiments, after the transdermal delivery device is applied to an administration site, a depot of the active agent forms in one or more skin layers. In some embodiments, the depot forms in one or more of the upper skin layers. Even after the delivery device is removed from the administration site, the active agent is therapeutically available for a period of time.

In some embodiments, the transdermal devices described herein provide sustained or continuous delivery of donepezil for at least about 6 hours to 4 days. In embodiments, the transdermal devices described herein provide sustained or continuous delivery of donepezil for at least about 6-72 hours, 6-48 hours, 6-24 hours, 6-12 hours, 12 hours to 4 days, 12-18 hours, 12-24 hours, 24 hours to 4 days, 24-72 hours, 24-48 hours, 48 hours to 4 days, 48-72 hours, or 72 hours to 4 days. It will be appreciated that the sustained or continuous delivery is achieved from a combination of delivery during the period the transdermal device is in contact with the skin and during the period subsequent to removal of the device from the skin when the drug is delivered from the depot that was created during the period of device contact with skin. The composition of the adhesive matrix in the transdermal devices described herein generate a donepezil depot in the subject, which provides benefits in terms of a decreased dosing frequency and/or increased patient compliance.

### IV. EXAMPLES

The following examples are illustrative in nature and are in no way intended to be limiting.

### Example 1

### Preparation of Adhesive Formulation Comprising Donepezil

An acrylate adhesive solution was prepared by dissolving an acrylic acid/vinyl acetate copolymer (DuroTak 387-2516) in a solvent to yield a solution.

An adhesive formulation was prepared by mixing the acrylate adhesive solution, levulinic acid, dimethyl sulfoxide, lauryl lactate, crosslinked polyvinylpyrrolidone, fumed silica (CAB-O-SIL®) until a homogeneous solution was formed. Donepezil in free base form was added to the solution and vortexed until dissolved. The adhesive formulation had a final composition as follows:

### Adhesive Formulation No. 1

| | | | |
|---|---|---|---|
| Active Agent | donepezil base | 10 | wt % |
| Adhesive Polymer(s) | acrylic acid/vinyl acetate | 40 | wt % |
| | copolymer | | |
| Solubility Enhancer | dimethyl succinate | 10-30 | wt % |
| Lipophilic Permeation | levulinic acid | 3 | wt % |
| Enhancer | | | |
| Matrix Modifier | crosslinked polyvinylpyrrolidone | 15 | wt % |
| | fumed silica | 7 | wt % |
| Skin Penetration | lauryl lactate | 2-4 | wt % |
| Enhancer | dimethyl sulfoxide | 0-5 | wt % |

### Example 2

### Preparation of Transdermal Device Comprising Donepezil

An adhesive matrix was prepared by coating the adhesive formulation of Example 1 onto a silicon-coated polyethylene terephthalate release liner at a wet thickness of 20 mils and then drying at about 70° C. for about 20 minutes. After drying, the adhesive drug formulation has a dry thickness of about 90 mm.

A backing layer (Scotchpak 9732) was laminated onto the adhesive matrix and transdermal devices of 10 cm² were die cut from the laminate.

### Example 3

### Preparation of Adhesive Formulation Comprising Donepezil

An adhesive formulation was prepared substantially as described in Example 1 to yield an adhesive formulation with the following composition:

### Adhesive Formulation No. 2

| | | | |
|---|---|---|---|
| Active Agent | donepezil base | 10 | wt % |
| Adhesive Polymer(s) | polyvinylpyrrolidone/vinyl | 5-15 | wt % |
| | acetate copolymer | | |
| | acrylic acid/vinyl acetate | 40 | wt % |
| | copolymer | | |
| Solubility Enhancer | dimethyl succinate | 10-30 | wt % |
| Lipophilic Permeation | levulinic acid | 3 | wt % |
| Enhancer | | | |
| Matrix Modifier | crosslinked polyvinylpyrrolidone | 15 | wt % |
| | fumed silica | 7 | wt % |
| Skin Penetration | lauryl lactate | 2-4 | wt % |
| Enhancer | dimethyl sulfoxide | 0-5 | wt % |

### Example 4

### Preparation of Adhesive Formulation Comprising Donepezil

An adhesive formulation was prepared substantially as described in Example 1 to yield an adhesive formulation with the following composition:

| | | | |
|---|---|---|---|
| Active Agent | donepezil base | 10 | wt % |
| Adhesive Polymer(s) | polyvinylpyrrolidone vinyl | 5-15 | wt % |
| | acetate copolymer | | |
| | acrylic acid/vinyl acetate | 40 | wt % |
| | copolymer | | |
| | acrylic acid copolymer | 5 | wt % |
| Solubility Enhancer | dimethyl succinate | 10-30 | wt % |
| Lipophilic Permeation | levulinic acid | 3 | wt % |
| Enhancer | | | |
| Matrix Modifier | crosslinked polyvinylpyrrolidone | 15 | wt % |
| | fumed silica | 7 | wt % |
| Skin Penetration | lauryl lactate | 2-4 | wt % |
| Enhancer | dimethyl sulfoxide | 0-5 | wt % |

### Example 5

### Preparation of Adhesive Formulation Comprising Donepezil

An adhesive formulation was prepared substantially as described in Example 1 to yield an adhesive formulation with the following composition:

### Adhesive Formulation No. 4

| Active Agent | donepezil base | 20 | wt % |
|---|---|---|---|
| Adhesive Polymer(s) | polyvinylpyrrolidone vinyl | 10-15 | wt % |
| | acetate copolymer | | |
| | acrylic acid/vinyl acetate | 40-60 | wt % |
| | copolymer | | |
| | polyisobutylene/polybutene | 5-20 | wt % |
| | mixture | | |
| Solubility Enhancer | | 0 | |
| Lipophilic Permeation | | 0 | |
| Enhancer | | | |
| Skin Penetration Enhancer | lauryl lactate | 2-6 | wt % |

### Example 6

### Preparation of Adhesive Formulation Comprising Donepezil

An adhesive formulation was prepared substantially as described in Example 1 to yield an adhesive formulation with the following composition:

### Adhesive Formulation No. 5

| | | | |
|---|---|---|---|
| Active Agent | donepezil base | 10 | wt % |
| Adhesive Polymer(s) | polyisobutylene/polybutene | 56 | wt % |
| | mixture | | |
| Solubility Enhancer | dimethyl succinate | 10-30 | wt % |
| Lipophilic Permeation | levulinic acid | 3 | wt % |
| Enhancer | | | |
| Matrix Modifier | fumed silica | 7 | wt % |
| Skin Penetration Enhancer | dimethyl sulfoxide | 0-5 | wt % |
| | lauryl lactate | 2-4 | wt % |

### Example 7

### Preparation of Adhesive Formulation Comprising Donepezil

An adhesive formulation was prepared substantially as described in Example 1 to yield an adhesive matrix:

### Adhesive Matrix Formulation No. 6

| | | | |
|---|---|---|---|
| Active Agent | donepezil base | 10 | wt % |
| Adhesive Polymer(s) | polyisobutylene/polybutene | 62 | wt % |
| | mixture | | |
| Solubility Enhancer | dimethyl succinate | 9.5 | wt % |
| Lipophilic Permeation Enhancer | levulinic acid | 3 | wt % |
| Matrix Modifier | fumed silica | 7 | wt % |
| Skin Penetration Enhancers | laurydone | 6 | wt % |
| | dimethyl sulfoxide | 2.5 | wt % |

### Example 8

### Evaluation of In Vitro Skin Flux

A transdermal patch with an active area of 5 cm² was prepared using the adhesive matrix of Example 7. The patch was applied to human cadaver skin in an in vitro apparatus, maintained at 32° C., and permeation of donepezil base into the receiving chamber of the apparatus was measured as a function of time. The transdermal patch was removed from the skin after 48 hours. Results of the study are in FIG. 2.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

All patents, patent applications, patent publications, and other publications mentioned herein are hereby incorporated by reference in their entirety. Where a patent, application, or publication contains express definitions, those definitions should be understood to apply to the incorporated patent, application or publication in which they are found and not to the present application unless otherwise indicated.

1.-11. (canceled)

12. A method for administering donepezil base, comprising:
contacting skin of a subject with a transdermal device comprising an adhesive matrix comprising 5-15 wt % donepezil base, about 9.5 wt % of dimethyl succinate, about 2.5 wt % dimethylsulfoxide, and about 6 wt % laurydone;
allowing the transdermal device to remain on the skin for 12-48 hours to create a donepezil depot in the skin; and
removing the transdermal device from the skin,
wherein, after said removing, donepezil base is administered to the subject from the donepezil depot for a period of at least about 6 hours.

13. The method of claim 12, wherein said adhesive matrix comprises about 10 wt % donepezil base.

14. (canceled)

15. The method of claim 12, wherein the donepezil base is adminstered from the donepezil for a period of 6-48 hours.

16. The method of claim 15, wherein a therapeutically effective amount of donepezil base is administered to the subject for at least about half of the period.

17. (canceled)

18. (canceled)

19. The method of claim 12, wherein the transdermal device remains on the skin for 48 hours.

20. The method of claim 19, wherein a skin flux of donepezil base is about 6 μg/cm² hr at 48 hours after contacting the skin of the subject with the transdermal device.

21. The method of claim 20, wherein the donepezil depot provides a sustained, continuous, or increasing skin flux of donepezil base for at least about 6 hours after the transdermal device is removed from the skin of the subject.

22. The method of claim 21, wherein the skin flux of donepezil base decreased to about 2.0 μg/cm² hr by about 72 hours after contacting the skin of the subject with the transdermal device.

23. The method of claim 22, wherein the skin flux of donepezil base is substantially as shown in FIG. 2.

24. The method of claim 12, wherein the adhesive matrix further comprises 2-5 wt % of a lipophilic permeation enhancer.

25. The method of claim 12, wherein the adhesive matrix further comprises a mixture of polyisobutylene and polybutene.
